(19) United States
(12) Patent Application Publication
(10) Pub. No.: US 2020/0302610 A1
(43) Pub. Date: Sep. 24, 2020

(54) MEDICAL IMAGE PROCESSING DEVICE, MEDICAL OBSERVATION DEVICE, METHOD OF PROCESSING IMAGE, AND COMPUTER READABLE RECORDING MEDIUM

(71) Applicant: Sony Olympus Medical Solutions Inc., Tokyo (JP)

(72) Inventor: Takaaki YAMADA, Kanagawa (JP)

(73) Assignee: Sony Olympus Medical Solutions Inc., Tokyo (JP)

(21) Appl. No.: 16/795,556

(22) Filed: Feb. 19, 2020

(30) Foreign Application Priority Data
Mar. 22, 2019 (JP) 2019-055737

(51) Int. Cl. G06T 7/11; G06T 7/00; G02B 21/36; G02B 21/00; A61B 1/00

(52) U.S. Cl. CPC G06T 7/11; G06T 7/0012; G02B 21/365; G06T 2207/20021; A61B 1/00009; G06T 2207/10056; G02B 21/0012

(57) ABSTRACT

A medical image processing device includes circuitry configured to: divide a color space into at least two areas by a hue when an instruction signal for changing a brightness level of an image signal captured by a medical observation device is received, the color space being a color space indicating a signal value of the image signal and being determined by a luminance signal and a color difference signal; and perform different signal processing on the image signal for each divided area of the color space.

This application claims priority from Japanese Application No. 2019-055737, filed on Mar. 22, 2019, the contents of which are incorporated by reference herein in its entirety.

### BACKGROUND

The present disclosure relates to a medical image processing device, a medical observation device, a method of processing image, and a computer readable recording medium.

In a medical device that captures an image of an object to be observed, a configuration including a switch with which a user such as a doctor adjusts brightness has been known (see, for example, JP 2006-15078 A). For example, when observing a deep part, the user brightens the entire screen by increasing a brightness level.

### SUMMARY

When a brightness level of the entire screen is increased, for example, a white area such as a nerve or a tumor in the deep part has a desired brightness, while a red part such as blood in an area that is in the vicinity of the deep part and is not the deep part becomes brighter than necessary to become a light color, such that it is not only impossible to reproduce an original color, but it is also difficult to distinguish from the white area.

There is a need for a medical image processing device, a medical observation device, a method of processing image, and a computer readable recording medium capable of providing an image with good color reproducibility by performing appropriate adjustment for each color component at the time of performing brightness adjustment.

According to one aspect of the present disclosure, there is provided a medical image processing device including circuitry configured to: divide a color space into at least two areas by a hue when an instruction signal for changing a brightness level of an image signal captured by a medical observation device is received, the color space being a color space indicating a signal value of the image signal and being determined by a luminance signal and a color difference signal; and perform different signal processing on the image signal for each divided area of the color space.

### BRIEF DESCRIPTION OF THE DRAWINGS

FIG. 1 is a view schematically illustrating a medical observation system according to an embodiment;

FIG. 2 is a block diagram illustrating a functional configuration of a medical observation device according to an embodiment;

FIG. 3 is a flowchart illustrating an outline of processing performed by a medical image processing device according to an embodiment; and

FIG. 4 is a flowchart illustrating an outline of processing performed by a medical image processing device according to a modification of an embodiment.

### DETAILED DESCRIPTION

Hereinafter, a mode (hereinafter referred to as an “embodiment”) for carrying out the present disclosure will be described with reference to the accompanying drawings.

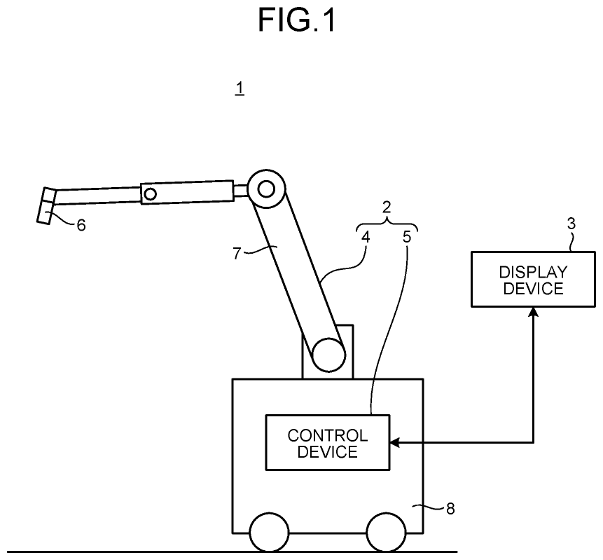

FIG. 1 is a view schematically illustrating a medical observation system according to an embodiment. A medical observation system 1 illustrated in FIG. 1 includes a medical observation device 2 and a display device 3.

The medical observation device 2 includes a microscope device 4 and a control device 5. The microscope device 4 has a function as an imaging device that captures an image of an object to be observed to acquire an image signal. The control device 5 has a function as a medical image processing device that performs image processing on the image signal captured by the microscope device 4. The medical observation device 2 according to the present embodiment is a microscope for a surgical operation.

The display device 3 receives an image signal for display generated by the control device 5 from the control device 5, and displays an image corresponding to the image signal. The display device 3 has a display panel made of liquid crystal or organic electro luminescence (EL).

An appearance configuration of the microscope device 4 will be described. The microscope device 4 includes a microscope unit 6 that magnifies a microstructure of an object to be observed and captures an image of the magnified microstructure, a support unit 7 that supports the microscope unit 6, a base unit 8 that holds a proximal end of the support unit 7 and incorporates the control device 5.

The microscope unit 6 has a cylindrical portion that forms a columnar shape. A cover glass is provided on an aperture surface at a lower end portion of a body portion (not illustrated). The cylindrical portion may be gripped by a user, and has a size at which it may be moved while being gripped by the user at the time of changing an imaging field of view of the microscope unit 6. Note that a shape of the cylindrical portion is not limited to a cylindrical shape, and may be a polygonal cylindrical shape.

The support unit 7 has a plurality of arm portions, and adjacent arm portions are rotatably connected to each other through joint portions. A transmission cable transmitting various signals between the microscope unit 6 and the control device 5 and a light guide transmitting illumination light generated by the control device 5 to the microscope unit 6 passes through a hollow portion formed inside the support unit 7.

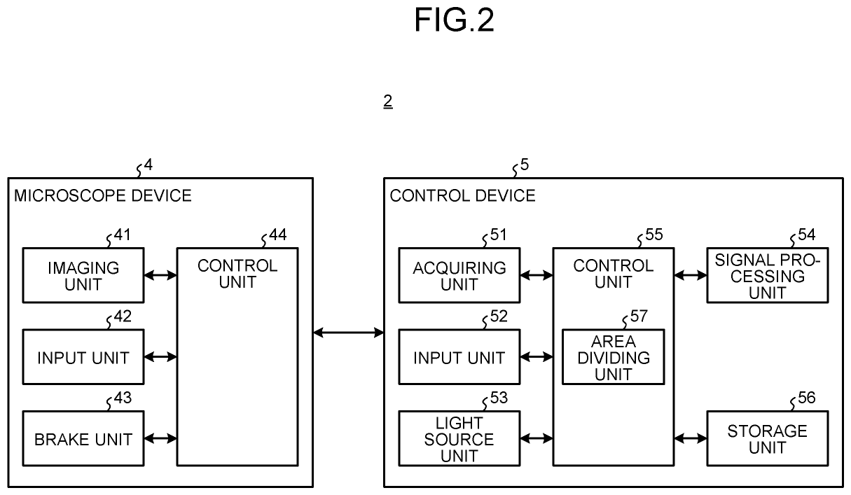

FIG. 2 is a block diagram illustrating a functional configuration of the medical observation device 2. First, a functional configuration of the microscope device 4 will be described. The microscope device 4 includes an imaging unit 41, an input unit 42, a brake unit 43, and a control unit 44.

The imaging unit 41 includes an optical system that has focus and zoom functions and an imaging element that generates an image signal by receiving an image of an object to be observed imaged by the optical system and performing photoelectric conversion. The optical system and the imaging element are provided inside the cylindrical portion of the microscope unit 6. The imaging element is configured using an image sensor such as a charge coupled device (CCD) or a complementary metal oxide semiconductor (CMOS). The image signal generated by the imaging unit 41 is transmitted to the control device 5 through the transmission cable. Note that the imaging unit 41 may include two imaging elements each having a predetermined parallax in each field of view and generate a three-dimensional image signal.

The input unit 42 receives an input such as an operation signal of the optical system of the imaging unit 41 or an operation signal of the brake unit 43. The input unit 42 is provided at a position where it may be operated by the user in a state where he/she holds the microscope unit 6, on a side surface of the cylindrical portion of the microscope unit 6. Note that the input unit 42 may receive an input of an instruction signal for changing a brightness level of an image displayed by the display device 3. In addition, the input unit 42 may be configured by further using a foot switch that the user may operate with his/her foot.

The brake unit 43 has a plurality of electromagnetic brakes provided, respectively, at a plurality of joint portions of the support unit 7. The electromagnetic brake is released when the input unit 42 receives an input of a release instruction. When the electromagnetic brake is released, one of two arms whose movement is regulated by the electromagnetic brake becomes rotatable with respect to the other of the two arms. Note that an actuator that assists the movement of the arm may be further provided at the joint portion.

The control unit 44 controls an operation of the microscope device 4 in cooperation with a control unit 55 of the control device 5. The control unit 44 is configured using at least one processor such as a central processing unit (CPU), a field programmable gate array (FPGA), and an application specific integrated circuit (ASIC).

Next, a functional configuration of the control device 5 will be described. The control device 5 includes an acquiring unit 51, an input unit 52, a light source unit 53, a signal processing unit 54, the control unit 55, and a storage unit 56.

The acquiring unit 51 acquires an image signal captured by the microscope device 4 and transmitted through the transmission cable. The image signal includes information related to image capturing, such as a focus position, a zoom, an exposure time, and an imaging time at the time of capturing an image.

The input unit 52 receives an input of various types of information including an instruction signal for changing a brightness level of the image. The input unit 52 is configured using a user interface such as a keyboard, a mouse, a touch panel, and a foot switch. Note that the input unit 52 may have at least some of the functions of the input unit 42 of the microscope device 4.

The light source unit 53 generates illumination light supplied to the microscope device 4 through the light guide. The light source unit 53 is configured using a discharge lamp such as a xenon lamp or a metal halide lamp, a solid light emitting element such as a light emitting diode (LED) or a laser diode (LD), a light emitting member such as a laser light source or a halogen lamp, or the like.

The signal processing unit 54 generates an image signal for display by performing signal processing on the image signal acquired by the acquiring unit 51, and outputs the generated image signal to the display device 3. The signal processing unit 54 is configured using at least one processor such as a CPU, an FPGA, and an ASIC.

The signal processing unit 54 performs conversion processing of a signal value on the image signal acquired by the acquiring unit 51. Specifically, the signal processing unit 54 converts a signal value (a signal value in an RGB color space) of the image signal acquired by the acquiring unit 51 into a signal value in a color space (hereinafter, referred to as a luminance-color difference color space) expressed using a luminance signal and two color difference signals. This conversion is defined by, for example, linear conversion. Specific examples of the luminance-color difference color space may include a YCbCr space or a YPbPr space.

The signal processing unit 54 performs signal processing for changing a luminance signal value in the luminance-color difference color space by different amounts for each area divided by an area dividing unit 57 of the control unit 55 to be described later. The signal processing unit 54 may change a luminance signal value in at least one of the divided areas. An amount of change in the luminance signal value may be stored in advance in the storage unit 56, or may be calculated by a predetermined calculation stored by the storage unit 56 based on information on an intensity of the illumination light generated by the light source unit 53 or an exposure time when the imaging unit 41 captures an image. The signal processing unit 54 generates an image signal for display by converting the signal value in the luminance-color difference color space into a signal value in an RGB space after changing the luminance signal value of the luminance-color difference color space.

The control unit 55 includes the area dividing unit 57 that divides the luminance-color difference color space into two areas according to a brightness level after being adjusted when the input unit 52 receives an input of an adjustment signal of the brightness level. For example, when the input unit 52 receives an input of an adjustment signal for increasing the brightness level, the control unit 55 divides the luminance-color difference color space into a first area including an area in which a hue is red and a second area other than the first area. A boundary between the first area and the second area divided by the area dividing unit 57 may be changed according to the brightness level after being adjusted.

The control unit 55 controls an operation of the control device 5, and controls an operation of the medical observation device 2 in cooperation with the control unit 44 of the microscope device 4. Specifically, for example, the control unit 55 controls light emission of the light source unit 53 by detecting the image signal acquired by the acquiring unit 51, and controls the exposure time in the imaging unit 41. In addition, the control unit 55 controls the display device 3 to display the image signal for display generated by the signal processing unit 54. The control unit 55 is configured using at least one processor such as a CPU, an FPGA, and an ASIC. Note that the signal processing unit 54 and the control unit 55 may be configured using a common processor.

The storage unit 56 stores information on the areas of the luminance-color difference color space divided by the area dividing unit 57 or information on an change amount for each hue when the signal processing unit 54 changes the luminance signal value in association with the brightness level. Note that the information stored by the storage unit 56 may be changeable by inputting settings by the user through the input unit 52.

The storage unit 56 stores various programs including a medical image processing program executed by the control device 5, and temporarily stores data that is being arithmetically processed by the control device 5. The storage unit 56 is configured using a read only memory (ROM), a random access memory (RAM), or the like. Note that the medical image processing program may be recorded on a computer-readable recording medium such as a hard disk, a flash memory, a CD-ROM, a DVD-ROM, or a flexible disc to be widely distributed.

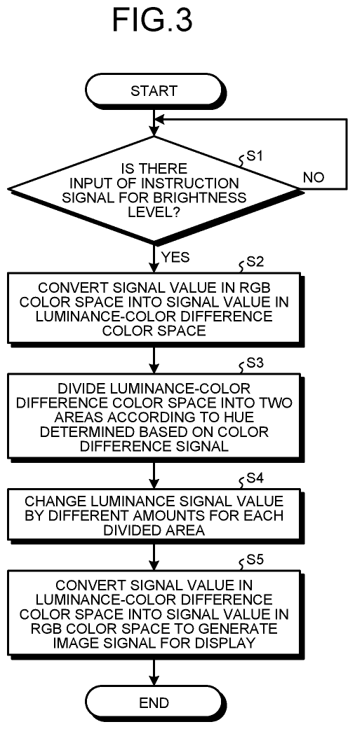

FIG. 3 is a flowchart illustrating an outline of processing performed by the control device 5. First, when the input unit 52 receives the input of the instruction signal for changing the brightness level (step S1: Yes), the signal processing unit 54 converts the signal value of the image signal, that is, the signal value in the RGB color space into the signal value in the luminance-color difference color space (step S2). When the input unit 52 does not receive the input of the instruction signal for changing the brightness level (step S1: No), the control device 5 repeats step S1.

After step S2, the area dividing unit 57 divides the luminance-color difference color space into two areas according to a hue determined based on a signal value of the color difference signal with reference to the storage unit 56 (step S3). For example, the area dividing unit 57 divides the luminance-color difference color space into a first area that is an area (red area) in which a hue includes red and a second area other than the first area.

Then, the signal processing unit 54 changes the luminance signal value by different amounts for each divided area with reference to the storage unit 56 (step S4). For example, when the brightness level is increased in a case of dividing the luminance-color difference color space into the first and second areas described above, the signal processing unit 54 decreases a luminance signal value of the first area, but does not change a luminance signal value of the second area. Thus, it is possible to decrease lightness of a red hue portion to adjust the red hue portion to an appropriate color and suppress a change in a color associated with a change in a brightness level, such that color reproducibility is improved and a contrast in a screen is increased.

Then, the signal processing unit 54 converts the signal value of the image signal from the signal value in the luminance-color difference color space into the signal value in the RGB color space to generate the image signal for display, and outputs the image signal for display to the display device 3 (step S5).

According to the embodiment described above, when the input of the instruction signal for changing the brightness level of the image signal is received, the color space determined by the luminance signal and the color difference signal is divided into the two areas by the hue, and different signal processing is performed on the divided two areas according to the brightness level. Therefore, it is possible to provide an image having good color reproducibility by performing appropriate adjustment for each color component at the time of performing brightness adjustment.

In addition, according to the present embodiment, when the instruction signal includes an instruction to increase the brightness level, the signal processing unit decreases a luminance signal value of an area that is one of the two areas and including a red hue portion. Therefore, it is possible to decrease the lightness of the red hue portion to adjust the red hue portion to the appropriate color and suppress the change in the color associated with the change in the brightness level, such that the color reproducibility is improved and the contrast in the screen is increased. As a result, for example, it becomes easy for the user to see a boundary between a tumor portion and a blood portion.

Modification

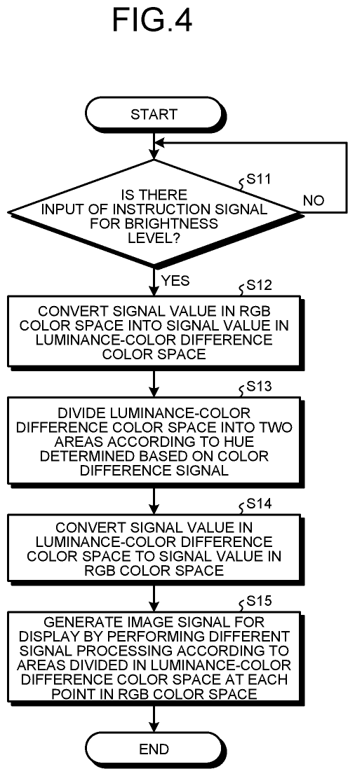

FIG. 4 is a flowchart illustrating an outline of processing performed by a medical image processing device (control device 5) according to a modification of the present embodiment. Steps S11 to S13 correspond, respectively, to steps S1 to S3 described with reference to FIG. 3.

In step S14, the signal processing unit 54 converts the signal value of the image signal from the signal value in the luminance-color difference color space to the signal value in the RGB color space (step S14).

Then, the signal processing unit 54 generates an image signal for display by performing different signal processing according to the areas divided in the luminance-color difference color space at each point in the RGB color space, and outputs the image signal to the display device 3 (step S15). For example, in the first area including the red hue portion, the signal value in RGB color space is changed so that the luminance signal value in the luminance-color difference color space is decreased, while in the second area, the signal value in RGB color space is changed so that the luminance signal value in the luminance-color difference color space is not changed.

The modification described above has the same effect as that of the embodiment.

Although the mode for carrying out the present disclosure has been described hereinabove, the present disclosure should not be limited only by the embodiment described above. For example, the medical observation system according to the present disclosure may be a medical endoscope system including a medical endoscope inserted into the object to be observed and capturing an image of the inside of the object to be observed.

Moreover, the area dividing unit 57 may divide the luminance-color difference color space into three areas or more than three areas, and the signal processing unit 54 may perform different signal processing on each of the divided areas.

According to the present disclosure, it is possible to provide an image with good color reproducibility by performing appropriate adjustment for each color component at the time of performing brightness adjustment.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical image processing device comprising
circuitry configured to:
divide a color space into at least two areas by a hue when an instruction signal for changing a brightness level of an image signal captured by a medical observation device is received, the color space being a color space indicating a signal value of the image signal and being determined by a luminance signal and a color difference signal; and
perform different signal processing on the image signal for each divided area of the color space.

2. The medical image processing device according to claim 1, wherein the circuitry is configured to convert a luminance signal value in the color space in at least one of the at least two areas.

3. The medical image processing device according to claim 2, wherein the circuitry is configured to decrease the luminance signal value in an area that is one of the at least two areas and includes a red hue portion when the instruction signal includes an instruction to increase the brightness level.

4. The medical image processing device according to claim 1, wherein the circuitry is configured to
convert a signal value in the color space into a signal value in an RGB color space, and
perform different signal processing on signal values in the RGB color space each corresponding to the at least two areas.

5. The medical image processing device according to claim 1, further comprising an input device configured to receive an input of the instruction signal for changing the brightness level of the image signal.

6. A medical observation device comprising:
an imaging device configured to capture an image of an object to be observed to acquire an image signal; and
a medical image processing device including a circuitry configured to
divide a color space into at least two areas by a hue when an instruction signal for changing a brightness level of the image signal captured by the imaging device is received, the color space being a color space indicating a signal value of the image signal and being determined by a luminance signal and a color difference signal, and
perform different signal processing on the image signal for each divided area of the color space.

7. A method of processing an image, the method comprising:
dividing a color space into at least two areas by a hue when an instruction signal for changing a brightness level of an image signal, the color space being a color space indicating a signal value of the image signal and being determined by a luminance signal and a color difference signal; and
performing different signal processing on the image signal for each divided area of the color space.

8. A non-transitory computer readable recording medium on which an executable program for processing an image, the program instructing a processor of a computer to execute:
dividing a color space into at least two areas by a hue when an instruction signal for changing a brightness level of an image signal, the color space being a color space indicating a signal value of the image signal and being determined by a luminance signal and a color difference signal; and
performing different signal processing on the image signal for each divided area of the color space.
